(19) United States
(12) Patent Application Publication
(10) Pub. No.: US 2025/0049646 A1
(43) Pub. Date: Feb. 13, 2025

(54) SOLID POWDER COSMETIC PRODUCT

(71) Applicant: SHISEIDO COMPANY, LTD., Chuo-ku, Tokyo (JP)

(72) Inventors: Ai TAKAMURA, Tokyo (JP); Kyoko JOICHI, Tokyo (JP); Yoshihiro NISHIYAMA, Tokyo (JP)

(73) Assignee: SHISEIDO COMPANY, LTD., Chuo-ku, Tokyo (JP)

(21) Appl. No.: 18/713,334

(22) PCT Filed: Dec. 9, 2022
(86) PCT No.: PCT/JP2022/045388

§ 371 Date: May 24, 2024

(30) Foreign Application Priority Data
Dec. 24, 2021 (JP) 2021-210094

(51) Int. Cl. A61K 8/02; A61K 8/19; A61K 8/25; A61K 8/29; A61K 8/36; A61K 8/44; A61Q 1/02; A61Q 1/12

(52) U.S. Cl. CPC A61K 8/022; A61K 8/19; A61K 8/25; A61K 8/29; A61K 8/361; A61K 8/44; A61Q 1/02; A61Q 1/12; A61K 2800/43; A61K 2800/612; A61K 2800/651

(57) ABSTRACT

The purpose of the present invention is to provide a solid powder composition having excellent moldability and impact resistance, the solid powder composition having good payoff, having no powdery feeling, and naturally taking on an even finish when configured as a cosmetic product. The present invention relates to a powder solid composition characterized by containing: (A) 0.1-30% by mass of an inorganic pigment treated with a surface treatment agent including an amino acid and/or a derivative thereof; and (B) 0.1-5% by mass of a metallic soap.

### TECHNICAL FIELD

The present invention relates to a solid powder composition and a cosmetic comprising the composition. More specifically, the present invention relates to a solid powder cosmetic that has excellent impact resistance and texture, and further has a natural and uniform finish and good color development.

### BACKGROUND ART

Solid powder cosmetic is a solid cosmetic generally obtained by filling and molding raw material prepared by adding oily components and the like to powder components into a dish-like container. Solid powder cosmetics are easy to carry; however, molded products may break if they are shaken or dropped while being carried. Moreover, since most of the components of solid powder cosmetics were powder components, there was a tendency to feel powderiness when applied to the skin. Further, there was a problem that when cosmetics were molded harder to improve impact resistance against vibration or dropping, the payoff of the cosmetics tended to become worse.

Patent Document 1 discloses a powdery cosmetic composition comprising a first non-spherical filler with an average particle size of less than 6 μm and being surface-treated with a surface treatment agent comprising at least one silicone oil, and a second filler being surface-treated with a surface treatment agent comprising at least one amino acid and/or a derivative thereof. Patent Document 1 indicates that by mixing the above two (non-spherical) fillers, even if a relatively large amount of spherical filler is contained, good hardness or compactability as well as good cosmetic properties (adhesion to the skin and spreadability on the skin) can be obtained. Specifically, the first non-spherical filler is silicone (dimethicone)-treated talc, and the second filler is mica treated with a mixture of palmitic acid, palmitoyl proline, sodium palmitoyl sarcosinate, and magnesium palmitoyl glutamate. However, the cosmetic of Patent Document 1 cannot achieve sufficient impact resistance if the first non-spherical filler surface-treated with silicone is not mixed (see Table 2 etc.).

On the other hand, a metallic soap is mixed in order to improve the impact resistance of solid powder cosmetics. Patent Document 2 discloses a solid powder cosmetic comprising more than 30 wt % of a metallic soap in combination with boron nitride, wherein the weight ratio of the metallic soap and boron nitride is within a predetermined range, thereby preventing caking and cracking, and allowing the solid powder cosmetic to spread smoothly and have a high moist feeling.

Patent Document 3 discloses a solid powder cosmetic comprising a metallic soap with a specific average particle size and particle size distribution, wherein the solid powder cosmetic is excellent in impact resistance, payoff, and the like. Patent Document 4 discloses a solid powder cosmetic comprising (a) metallic soap fine particles with a number average particle size of 1 to 3 μm and (b) partially-crosslinked organopolysiloxane, wherein the solid powder cosmetic is excellent in impact resistance and texture (spreadability at the time of use and a good moist feeling).

However, there was a problem that if a pigment surface-treated with silicone, such as the first non-spherical filler that is indispensable component in the cosmetic of Patent Document 1, coexists with a metallic soap in a solid powder cosmetic, the payoff of the solid powder cosmetic becomes worse.

### CITATION LIST

### Patent Documents

Patent Document 1: JP-T 2013-544830
Patent Document 2: JP-B 2993627
Patent Document 3: JP-B 4051787
Patent Document 4: JP-A 2018-168145

### SUMMARY OF INVENTION

### Technical Problem

An object of the present invention is to provide a solid powder composition that has excellent moldability and impact resistance, and that is good in payoff, has no powderiness (no powdery feeling), and has a natural and uniform finish when used as a cosmetic.

### Solution to Problem

As a result of extensive research to achieve the above object, the present inventors have found that mixing a metallic soap together with an inorganic pigment treated with a surface treatment agent comprising an amino acid or a derivative thereof results in solid powder cosmetics that have excellent impact resistance and that are good in payoff and have good texture and a good finish when used as cosmetics, as well as that are excellent in the chromogenic properties of the inorganic pigment. The present invention has thus been completed.

Specifically, the present invention provides a powder solid composition characterized by comprising:
(A) 0.1 to 30% by mass of an inorganic pigment treated with a surface treatment agent comprising an amino acid and/or a derivative thereof; and
(B) 0.1 to 5% by mass of a metallic soap.

### Advantageous Effect of Invention

The composition of the present invention can be formed into a solid powder cosmetic that has excellent impact resistance, is good in payoff (i.e., pickup of a cosmetic by an applicator such as finger, brush or the like), has no powderiness during use, and has a natural and uniform finish. In addition, the composition of the present invention is also excellent in the chromogenic properties of the inorganic pigment (white pigment and/or color pigment).

### DESCRIPTION OF EMBODIMENTS

(A) Inorganic Pigment Treated with a Surface Treatment Agent Comprising an Amino Acid and/or a Derivative Thereof

The first essential component in the solid powder composition of the present invention (hereinafter also referred to simply as “the composition”) is an inorganic pigment treated with a surface treatment agent comprising an amino acid and/or a derivative thereof (hereinafter also referred to as “the amino acid-treated inorganic pigment” or “component A”).

“Inorganic pigments” used in cosmetics generally include “extender pigments” including talc, mica, sericite, synthetic phlogopite, and barium sulfate, “white pigments” typified by titanium oxide and zinc oxide, and “color pigments” including iron oxides, such as red iron oxide (colcothar), yellow iron oxide, and black iron oxide. In the present invention, the “inorganic pigment” that is used as a substrate for the “amino acid-treated inorganic pigment” (component A) is preferably a “white pigment” and/or a “color pigment.” Mixing chromogenic white pigments and/or color pigments surface-treated with an amino acid and/or a derivative thereof results in cosmetics with excellent chromogenic properties when applied. The “white pigment” is preferably titanium oxide, and the “color pigment” is preferably iron oxide.

The “inorganic pigment” that serves as a substrate for the “amino acid-treated inorganic pigment” has a so-called “pigment grade” size, and is generally an inorganic pigment with an average particle size (median diameter) of 100 to 500 nm. In particular, an inorganic pigment with an average particle size of 200 nm or more is preferably used. Titanium oxide fine particles and zinc oxide fine particles with an average particle size of several tens of nanometers, which are used as UV scattering agents, are not included in the “inorganic pigment” in the present invention.

The inorganic pigment in the present invention is surface-treated with a “surface treatment agent comprising an amino acid and/or a derivative thereof.” The “amino acid” is not particularly limited, and examples include glutamic acid, aspartic acid, lysine, and the like. The “derivative of the amino acid” is preferably “N-acyl amino acid or a salt thereof.”

The “acyl group” of the N-acyl amino acid is preferably a group in which a hydroxyl group is removed from a C₁₂-20 saturated fatty acid, and examples include a stearoyl group, a lauroyl group, and the like. The “salt” can be selected from salts of alkali metals such as sodium and potassium, alkaline earth metal salts, and the like, but is preferably a sodium salt. Preferred examples of the N-acyl amino acid or a salt thereof include lauroyl lysine, disodium N-stearoyl glutamate, sodium N-lauroyl glutamate, and sodium lauroyl aspartate.

The “surface treatment agent comprising an amino acid and/or a derivative thereof” used in the “amino acid-treated inorganic pigment” (component A) in the composition (cosmetic) of the present invention may contain at least one amino acid or a derivative thereof, and examples include a surface treatment agent composed of lauroyl lysine (also referred to as “LL treatment agent”). On the other hand, an inorganic pigment surface-treated with a surface treatment agent comprising at least two treatment agents is particularly preferred, and one of the at least two treatment agents is preferably N-acyl amino acid or a salt thereof.

In the surface treatment agent comprising at least two treatment agents, the “other treatment agent” other than the “N-acyl amino acid or a salt thereof” mentioned above is preferably selected from an “amino acid,” “an ester,” and “a metal complex of ester.” The “amino acid” is not particularly limited, and is preferably selected from aspartic acid, glutamic acid, and lysine. The “ester” is a compound in which a C₈-12 monovalent or divalent fatty acid and a C₁₂-20 saturated aliphatic alcohol are bonded via an ester bond, and the alkyl chains of the fatty acid and aliphatic alcohol may be linear or branched. In particular, isostearyl sebacate is preferably used. The “metal complex of ester” is a compound in which an ester is coordinately bonded to a metal atom, and preferred examples include isopropyl titanium triisostearate.

Specific examples of the surface treatment agent comprising at least two treatment agents include a surface treatment agent comprising sodium N-lauroyl glutamate and lysine (also referred to as “ASL treatment agent”), a surface treatment agent comprising disodium N-stearoyl glutamate and isostearyl sebacate (also referred to as “NHS treatment agent”), and a surface treatment agent comprising sodium lauroyl aspartate and isopropyl titanium triisostearate (also referred to as “ASI treatment agent”). In the present invention, an inorganic pigment surface-treated with an ASL treatment agent is particularly preferably used.

The composition of the present invention may be mixed with one or more amino acid-treated inorganic pigments (components A), and the total mixing amount thereof is 0.5 to 30% by mass, and preferably 1 to 20% by mass, based on the total amount of the cosmetic. If the mixing amount is less than 0.5% by mass, sufficient color development cannot be obtained. If the mixing amount exceeds 30% by mass, the uniformity of finish is lost, and the payoff of the cosmetic tends to be worse.

### (B) Metallic Soap

The second essential component in the composition of the present invention is a “metallic soap” (hereinafter also referred to as “component B”). “Metallic soaps” used in cosmetics are generally understood to be higher fatty acid salts of non-alkali metals, such as calcium, zinc, and magnesium. In the present invention, it is preferable to use a C₁₂-22 fatty acid calcium salt or a C₁₂-22 fatty acid magnesium salt.

Examples of the fatty acid that constitutes the metallic soap (component B) in the present invention include lauric acid, myristic acid, myristoleic acid, palmitic acid, palmitoleic acid, stearic acid, oleic acid, linoleic acid, arachidic acid, behenic acid, erucic acid, hydroxystearic acid, epoxystearic acid, and the like; particularly preferred among these is myristic acid. When a mixture of two or more fatty acids is used, the myristic acid content in the fatty acid mixture is preferably 50% by mass or more, more preferably 60% by mass or more, and even more preferably 70% by mass or more.

The metallic soaps preferably used in the present invention are calcium, zinc, and magnesium salts of C₁₂₋₂₂ fatty acids, and particularly calcium myristate, zinc myristate, and magnesium myristate.

Although the size and shape of the metallic soap in the present invention are not particularly limited, a plate-like shape having an average particle size (median diameter) of 4.0 to 40.0 μm is generally preferred.

The C₁₂₋₂₂ fatty acid calcium salt is particularly preferably fatty acid calcium salt particles that satisfy the following conditions (I) to (III) (hereinafter referred to as “the specific fatty acid calcium salt particles”).
(I) a median diameter (D50) of 4.0 μm to 15.0 μm;
(II) a particle size digest A represented by the following formula (1) satisfying the relationship A≤2.0, and preferably 0.5≤A≤1.8:

$$particle size digest A = ( D 90 - D 10 ) / D 50 \qquad ( 1 )$$

[provided that 4.0≤D50≤15.0,
D10 represents the 10% cumulative diameter (μm) based on the volume of the fatty acid calcium salt,
D50 represents the median diameter (μm) based on the volume of the fatty acid calcium salt, and
D90 represents the 90% cumulative diameter (μm) based on the volume of the fatty acid calcium salt]; and
(III) an average thickness of 350 to 800 nm, and preferably 400 to 700 nm.

The specific fatty acid calcium salt particles preferably have a plate-like shape.

Each parameter that determines the “particle size digest A” defined by the above formula (1) is calculated from, for example, the particle size measured by a Microtrac laser diffraction method. The Microtrac laser diffraction method is a method for determining particle size distribution using scattered light obtained by irradiating particles with laser light. For example, the measurement can be carried out by using Microtrac MT-3000 produced by Nikkiso Co., Ltd.

When the cumulative curve of the particle size of the measured particles is calculated using the total volume of the particle population as 100%, the particle sizes at the points where the cumulative curves are 10%, 50%, and 90% are defined as 10% cumulative diameter (D10) (μm), 50% median diameter (D50; median diameter) (μm), and 90% cumulative diameter (D90) (μm), respectively.

Further, the thickness of the particles is a value of the length (thickness) of the side surface when the side with the largest area is the front in the fatty acid calcium salt particles. The average thickness of the particles is the average value determined by measuring the length of the side surface for 10 particles when the side with the largest area is the front in the fatty acid calcium salt particles. The average thickness of the particles is a value measured based on two-dimensional projection images (e.g., SEM photographs) of the particles.

The specific fatty acid calcium salt particles can be prepared by a double decomposition method in which a fatty acid alkali compound salt obtained by reacting a monovalent alkali compound with a C₁₂₋₂₂ fatty acid is reacted with a divalent calcium salt in an aqueous solution, as with general fatty acid calcium salts. Examples of the monovalent alkali compound as a raw material for the fatty acid alkali compound salt include hydroxides of alkali metals (sodium, potassium, etc.), and amines such as ammonia, monoethanolamine, diethanolamine, and triethanolamine. Examples of the divalent calcium salt include calcium chloride, calcium acetate, and the like.

In the above production method, the concentration of the fatty acid alkali compound salt, the temperature in the reaction of the fatty acid alkali compound salt and the calcium salt, and the dropping speed of dropping a calcium salt-containing aqueous solution to a fatty acid alkali compound salt-containing aqueous solution can be each appropriately controlled to adjust the particle size and particle thickness, thereby obtaining specific fatty acid calcium salt particles. For example, when a calcium salt-containing aqueous solution is added dropwise to a fatty acid alkali compound salt-containing aqueous solution, the dropping speed is preferably 0.005 to 0.8 mol/mol, and more preferably 0.01 to 0.5 mol/mol, per unit time, whereby the exchange reaction between alkali and calcium proceeds mildly, and fatty acid calcium salt particles having an appropriate thickness can be obtained. The unit “mol/mol” of the calcium salt dropped is the number of moles of the calcium salt per mol of the fatty acid alkali compound salt.

The fatty acid calcium salt cake obtained by the above reaction is separated by a dehydrator, a filter press, or the like to reduce the water content, and then dried by a rotary dryer, an airflow dryer, a vented shelf dryer, a spray dryer, a fluidized bed dryer, or the like, thereby obtaining specific fatty acid calcium salt particles. In this drying process, the particle size distribution and thickness can also be adjusted to be within appropriate ranges. For example, it is preferable to set the drying temperature within the range of (α−40) ° C.≤α≤(α+5° C.) relative to the contained water evaporation peak top temperature («° C.) of the fatty acid calcium salt. The contained water evaporation peak top temperature refers to the top peak of the temperature range at which residual water that cannot be removed by the above drying process contained in the fatty acid calcium salt begins to desorb. For example, in a heat absorption graph of calcium myristate obtained by differential thermal analysis (DSC), the contained water evaporation peak top temperature is 110.3° C. Therefore, in the case of calcium myristate, drying in a temperature range of 70° C. or higher and 115° C. or lower results in particles having an appropriate thickness without causing adhesion between the particles. Alternatively, dried particles can be classified using a sieve of 100 mesh, 200 mesh, 330 mesh, or the like for adjustment.

The C₁₂₋₂₂ fatty acid magnesium salt is particularly preferably fatty acid magnesium salt particles that satisfy the following conditions (I) and (II) (hereinafter referred to as “the specific fatty acid magnesium salt particles”).
(I) An aspect ratio of 1.0 or more and 2.0 or less, preferably 1.0 or more and 1.6 or less, and more preferably 1.0 or more and 1.5 or less, which is represented by the following formula (2):
aspect ratio=major axis diameter (μm) of particles/minor axis diameter (μm) of particles . . . (2); and
(II) an average thickness of 250 to 600 nm, preferably 280 to 450 nm, and more preferably 300 to 450 nm.

The specific fatty acid magnesium salt particles preferably have a plate-like shape.

The aspect ratio defined by the above formula (2) is a value obtained by dividing the major axis diameter of the fatty acid magnesium salt particles by the minor axis diameter. The “major axis diameter” is the length of the major axis of the particle, and specifically corresponds to the particle width at which the distance between two parallel lines that sandwich the particle is maximum. The “minor axis diameter” is the length of the minor axis of the particle, and specifically corresponds to the particle width measured on a straight line passing through the midpoint of the major axis and perpendicular to the major axis.

The average thickness of the particles is the same as the definition described above regarding the specific fatty acid calcium salt particles.

The specific fatty acid magnesium salt particles in the present invention preferably have a particle index of 1.5 or more and 8.0 or less, more preferably 1.5 or more and 6.0 or less, and even more preferably 2.0 or more and 5.0 or less, which is defined by the following formula (3):

$$particle index = [ \text{} ( major axis diameter ( μ m ) of particles / minor axis diameter ( μ m ) of particles ) / average thickness ( nm ) of particles ] \times 1000 \qquad ( 3 )$$

Because the particle index is within the above range, the particles as a cosmetic can be easily applied uniformly to the skin and can maintain the texture immediately after application for a longer period of time.

Further, the specific fatty acid magnesium salt particles more preferably satisfy that the median diameter is within the range of 10.0 to 40.0 μm and that the particle size digest A defined by the above formula (1) is 2.5 or less. Satisfying these conditions means that the particles have a narrow particle size distribution. This allows for uniform presence in cosmetics, and the texture of cosmetics is more likely to be improved stably.

The specific fatty acid magnesium salt particles can be produced according to the production method of the specific fatty acid calcium salt particles described above. In that production method, it is possible to adjust the parameters such as particle size, and the dried final product can be classified to obtain particles that satisfy the conditions regarding particle size.

The composition of the present invention comprises (A) an amino acid-treated inorganic pigment and (B) a metallic soap as essential components, and is particularly suitable for use for a solid powder cosmetic. Therefore, the composition of the present invention may be mixed with any other components that can be used in solid powder cosmetics within the range that does not impair the effects of the present invention.

### (C) High-Viscosity Oil

It is preferable to mix a high-viscosity oil (also referred to as “component C”) from the viewpoint of maintaining and improving the impact resistance of the solid powder composition. The “high-viscosity oil” in the present invention refers to a liquid or semi-solid oil with a viscosity at 30° C. of 1000 cps (mPa·s) or more, and preferably 8000 to 500000 cps (BL viscometer, rotor No. 3, 12 rpm). The (C) high-viscosity oil is preferably selected from non-silicone oils, and an ester oil or a hydrocarbon oil is preferably used.

Specific examples of the (C) high-viscosity oil include isostearic acid esters including diisostearyl malate, dipentaerythritol monostearate, sucrose tetraisostearate, and isostearic acid esters of polyglycerin, such as diglycerin diisostearate, triglycerin diisostearate, and decaglycerin decaisostearate; heavy liquid isoparaffins including hydrogenated copolymers of isobutene and n-butene, and polybutene; petrolatum, and the like. These may be mixed singly or in combination of two or more.

The amount of the high-viscosity oil (component C) in the composition of the present invention is not particularly limited, and is generally 3% by mass to 20% by mass, and preferably 5 to 15% by mass. If the amount is less than 3% by mass, the effect of mixing the high-viscosity oil (e.g., improvement in impact resistance) is less likely to be obtained. If the amount exceeds 20% by mass, caking tends to occur easily.

### (D) Spherical Silica Powder

Texture as a cosmetic is further improved by mixing a spherical silica powder (also referred to as “component D”).

Although the spherical silica (silicic anhydride) powder is not particularly limited, porous, hollow, or non-porous silica with an average particle size (median diameter) of 1 to 50 μm can be used.

The amount of the spherical silica powder (component D) in the composition of the present invention is generally 0.1 to 10% by mass, and preferably 1 to 8% by mass. If the mixing amount is less than 0.1% by mass, the effect of mixing the spherical silica powder (e.g., improvement in texture) is less likely to be obtained. If the mixing amount exceeds 10% by mass, powderiness tends to be felt.

### (E) Silicone Elastomer

When a silicone elastomer (also referred to as “component E”) is mixed in the composition of the present invention, the finish of the cosmetic when applied becomes more uniform and smooth. The silicone elastomer is not limited, and examples include polyether-modified silicone elastomers such as a (dimethicone/(PEG-10/15)) crosspolymer, polyglycerin-modified silicone elastomers such as a (dimethicone/polyglycerin-3) crosspolymer, and the like.

The amount of the silicone elastomer (component E) in the composition of the present invention is generally 0.5 to 10% by mass, and preferably 1 to 8% by mass. If the mixing amount is less than 0.5% by mass, the effect of mixing the silicone elastomer (e.g., improvement in finish) is less likely to be obtained. If the mixing amount exceeds 10% by mass, powderiness tends to be felt.

Examples of other optional components include, but are not limited to, powder components that do not belong to the above components A to E, surfactants, moisturizers, polymers, dyes, lower alcohols, polyhydric alcohols, antioxidants, UV absorbers, UV scattering agents, various beauty components, antibacterial agents, preservatives, pH adjusters, fragrances, and the like.

The powder components that do not belong to the above components A to E include inorganic pigments other than the component A (i.e., surface-untreated inorganic pigments and inorganic pigments treated with a surface treatment agent that does not contain an amino acid or a derivative thereof; the inorganic pigments as mentioned herein include white pigments, color pigments, ad extender pigments), and pearlescent powders.

Examples of pearlescent powders (pearl agents) include titanium mica, iron oxide-coated titanium mica, carmine-coated titanium mica, carmine/iron blue-coated titanium mica, iron oxide/carmine-treated titanium mica, iron blue-treated titanium mica, iron oxide/iron blue-treated titanium mica, chromium oxide-treated titanium mica, black titanium oxide-treated titanium mica, acrylic resin-coated aluminum powder, silica-coated aluminum powder, titanium oxide-coated mica, titanium oxide-coated bismuth oxychloride, titanium oxide-coated talc, colored titanium oxide-coated mica, titanium oxide-coated synthetic mica, titanium oxide-coated silica, titanium oxide-coated alumina, titanium oxide-coated glass flakes, polyethylene terephthalate/polymethyl methacrylate laminated film powder, bismuth oxychloride, fish scale foil, and the like.

The composition of the present invention is particularly suitable for use as a solid powder cosmetic with excellent impact resistance and texture. Examples of forms provided as solid powder cosmetics include make-up cosmetics, such as foundation, concealer, face powder, control color, eye shadow, eye liner, cheek color, body powder, perfume powder, and baby powder.

### EXAMPLES

The present invention will be described in more detail below with reference to Examples; however, the present invention is not limited by these Examples. The mixing amount is expressed by % by mass, unless otherwise specified.

Solid powder cosmetics were prepared according to a conventional method with the formulations shown in Tables 1 and 2 below. For the cosmetic of each example, payoff of the cosmetic, no powderiness, finish uniformity, and chromogenic properties were sensory evaluated by trained panelists. In addition, impact resistance was evaluated by a drop test. The evaluation results are shown according to the following criteria.
A+: extremely excellent
A: excellent
B: good
C: slightly inferior
D: inferior

TABLE 1

| | Example 1 | Example 2 | Example 3 | Example 4 | Example 5 |
|---|---|---|---|---|---|
| Talc | Remaining | Remaining | Remaining | Remaining | Remaining |
| | amount | amount | amount | amount | amount |
| Mica | 5 | 5 | 5 | 5 | 5 |
| Specific calcium myristate particles | 2 | — | — | — | 2 |
| Specific magnesium myristate particles | — | 2 | — | — | — |
| Zinc myristate (*1) | — | — | 2 | — | — |
| Magnesium myristate (*2) | — | — | — | 2 | — |
| Zinc oxide | 1 | 1 | 1 | 1 | 1 |
| Silica | 5 | 5 | 5 | 5 | 5 |
| Glitter powder | 30 | 30 | 30 | 30 | 30 |
| Amino acid-treated titanium oxide | 1 | 1 | 1 | 1 | 1 |
| Amino acid-treated red iron oxide | 1 | 1 | 1 | 1 | 1 |
| Amino acid-treated yellow iron oxide | 1 | 1 | 1 | 1 | 1 |
| Amino acid-treated black iron oxide | 1 | 1 | 1 | 1 | 1 |
| Silicone elastomer | — | — | — | — | 2 |
| Vaseline | 5 | 5 | 5 | 5 | 5 |
| Trimethylolpropane triethylhexanoate | 4 | 4 | 4 | 4 | 4 |
| Diisostearyl malate | 2 | 2 | 2 | 2 | 2 |
| Preservative | q.s. | q.s. | q.s. | q.s. | q.s. |
| Antioxidant | q.s. | q.s. | q.s. | q.s. | q.s. |
| Total | 100 | 100 | 100 | 100 | 100 |
| Payoff of the cosmetic | A+ | A+ | A+ | A+ | A+ |
| No powderiness | A+ | A | B | B | A+ |
| Finish uniformity | A | A | B | B | A+ |
| Impact resistance | A+ | A | A | A | A+ |
| Chromogenic properties | A+ | A | A | A | A+ |

*1 Zinc myristate that does not satisfy at least one of the conditions for specific zinc myristate
*2 Mg myristate that does not satisfy at least one of the conditions for specific Mg myristate

TABLE 2

| | Comparative | Comparative | Comparative | Comparative | Comparative |
|---|---|---|---|---|---|
| | Example 1 | Example 2 | Example 3 | Example 4 | Example 5 |
| Talc | Remaining | Remaining | Remaining | Remaining | Remaining |
| | amount | amount | amount | amount | amount |
| Mica | 5 | 5 | 5 | 5 | 5 |
| Specific calcium myristate | 2 | — | — | — | — |
| particles | | | | | |
| Specific magnesium myristate | — | — | — | — | 2 |
| particles | | | | | |
| Zinc oxide | 1 | 1 | 1 | 1 | 1 |
| Silica | 5 | 5 | 5 | 5 | 5 |
| Glitter powder | 30 | 30 | 30 | 30 | 30 |
| Amino acid-treated titanium | — | 1 | 20 | — | — |
| oxide | | | | | |
| Silicone-treated titanium oxide | 1 | — | — | 1 | 1 |
| Amino acid-treated red iron oxide | — | 1 | 5 | — | — |
| Silicone-treated red iron oxide | 1 | — | — | 1 | 1 |
| Amino acid-treated yellow iron | — | 1 | 5 | — | — |
| oxide | | | | | |
| Silicone-treated yellow iron oxide | 1 | — | — | 1 | 1 |
| Amino acid-treated black iron | — | 1 | 5 | — | — |
| oxide | | | | | |
| Silicone-treated black iron oxide | 1 | — | — | 1 | 1 |
| Vaseline | 5 | 5 | 5 | 5 | 5 |
| Trimethylolpropane | 4 | 4 | 4 | 4 | 4 |
| triethylhexanoate | | | | | |
| Diisostearyl malate | 2 | 2 | 2 | 2 | 2 |
| Preservative | q.s. | q.s. | q.s. | q.s. | q.s. |
| Antioxidant | q.s. | q.s. | q.s. | q.s. | q.s. |
| Total | 100 | 100 | 100 | 100 | 100 |
| Payoff of the cosmetic | C | A+ | C | A+ | C |
| No powderiness | A | D | A | D | A |
| Finish uniformity | A | D | D | D | A |
| Impact resistance | A+ | D | A | D | A |
| Chromogenic properties | C | A | A | C | C |

As shown in Table 1, Examples 1 to 5, in which (A) an amino acid-treated inorganic pigment and (B) a metallic soap were mixed, satisfied all of the evaluation items. In particular, in Examples 1, 2, and 5, in which specific calcium myristate particles or specific magnesium myristate particles were used as the metallic soap, “no powderiness” and “finish uniformity” were further improved in comparison to Examples 3 and 4, in which metallic soaps other than those above were mixed. In particular, Example 5, in which a silicone elastomer was mixed, was evaluated as extremely excellent in all items.

As shown in Table 2, Comparative Examples 1 and 5, in which the amino acid-treated inorganic pigment was replaced by a silicone-treated inorganic pigment, “payoff of the cosmetic” and “chromogenic properties” were insufficient. Further, in Comparative Example 4, in which metallic soaps were removed, texture (powderiness) and finish were poor and impact resistance was insufficient. On the other hand, in Comparative Example 2, in which an amino acid-treated inorganic pigment was mixed but a metallic soap was not mixed, texture (powderiness) and finish were poor and impact resistance was insufficient, as in Comparative Example 4. In Comparative Example 3, in which the mixing amount of the amino acid-treated inorganic pigment in Comparative Example 2 was increased to 35% by mass, texture and impact resistance were improved, whereas uniform finish was not obtained.

1. A powder solid composition comprising:
(A) 0.1 to 30% by mass of an inorganic pigment treated with a surface treatment agent comprising an amino acid and/or a derivative thereof; and
(B) 0.1 to 5% by mass of a metallic soap.

2. The composition according to claim 1, wherein the inorganic pigment is selected from the group consisting of iron oxide and titanium oxide.

3. The composition according to claim 1, wherein the amino acid and/or a derivative thereof in the surface treatment agent comprises N-acyl amino acid.

4. The composition according to claim 1, wherein the amino acid is selected from the group consisting of glutamic acid, aspartic acid, and lysine.

5. The composition according to claim 3, wherein the N-acyl amino acid is lauroyl amino acid or stearoyl amino acid.

6. The composition according to claim 1, wherein the (B) metallic soap is a C₁₂₋₂₂ fatty acid calcium salt or magnesium salt.

7. The composition according to claim 6, wherein the C₁₂₋₂₂ fatty acid calcium salt is fatty acid calcium salt particles that satisfy the following conditions:
a median diameter of 4.0 μm to 15.0 μm;
a particle size digest A represented by the following formula (1) satisfying the relationship A≤2.0:
$$particle size digest A = ( D 90 - D 10 ) / D 50 \qquad ( 1 )$$
[provided that 4.0≤D50≤15.0,
D10 represents the 10% cumulative diameter (μm) based on the volume of the fatty acid calcium salt,
D50 represents the median diameter (μm) based on the volume of the fatty acid calcium salt, and
D90 represents the 90% cumulative diameter (μm) based on the volume of the fatty acid calcium salt]; and
an average thickness of 350 to 800 nm.

8. The composition according to claim 6, wherein the C₁₂₋₂₂ fatty acid magnesium salt is fatty acid magnesium salt particles that satisfy the following conditions:
an aspect ratio of 1.0 or more and 2.0 or less, which is represented by the following formula (2):
$$aspect ratio = major axis diameter ( μ m ) of particles / minor axis diameter ( μ m ) of particles ; \qquad ( 2 )$$
and
an average thickness of 250 to 600 nm.

9. The composition according to claim 1, further comprising (C) a high-viscosity oil.

10. The composition according to claim 1, further comprising (D) a spherical silica powder.

11. The composition according to claim 1, further comprising (E) a silicone elastomer.

12. A powder solid cosmetic comprising the composition according to claim 1.
